(19) United States
(12) Patent Application Publication
(10) Pub. No.: US 2026/0086034 A1
(43) Pub. Date: Mar. 26, 2026

(54) MEASUREMENT DEVICE AND MEASUREMENT METHOD

(71) Applicant: HAMAMATSU PHOTONICS K.K., Hamamatsu-shi (JP)

(72) Inventors: Kimiko KAZUMURA, Shizuoka (JP); Yoshinao NAGAI, Shizuoka (JP)

(73) Assignee: HAMAMATSU PHOTONICS K.K., Hamamatsu-shi (JP)

(21) Appl. No.: 19/331,748

(22) Filed: Sep. 17, 2025

(30) Foreign Application Priority Data
Sep. 20, 2024 (JP) 2024-163903

(51) Int. Cl. G01N 21/64; G01N 33/52

(52) U.S. Cl. CPC G01N 21/6428; G01N 33/52; G01N 2021/6439

(57) ABSTRACT

A measurement device including: a support unit for supporting a first container accommodating therein a first solution containing an indicator and frozen, the indicator being to react with a component generated from a biological sample; a heater for heating the first solution in the first container in a support state where the support unit supports the first container; a first addition unit for, in the support state, adding a second solution containing a stimulant to the first solution in the first container, the stimulant being to activate a function of the biological sample; and a second addition unit for, in the support state, adding the biological sample to the first solution in the first container.

### TECHNICAL FIELD

The present disclosure relates to a measurement device and a measurement method.

### BACKGROUND

Thus far, a technology that measures characteristics of a biological sample has been known (see, for example, Japanese Unexamined Patent Publication No. 2019-213464). In such a technology, characteristics of a biological sample collected from a subject (for example, a human body) may be measured by a method in which the biological sample is added to a preparation solution prepared by introduction of a plurality of reagents into a container and then light generated in an accommodation space of the container is measured.

### SUMMARY

In a technology like that described above, at least one of a plurality of reagents may be likely to deteriorate in a state of normal temperature; in this case, it is desirable that operations such as preparation of a preparation solution and introduction of a preparation solution into a container be performed immediately before measurement of light and in the vicinity of the subject. However, if these operations are performed in the vicinity of the subject, the operations may be complicated.

An object of the present disclosure is to provide a measurement device and a measurement method that enable simple operations.

A measurement device according to the present disclosure is [1] “A measurement device used for measurement of a characteristic of a biological sample, the measurement device including: a support unit for supporting a first container configured to accommodate therein a first solution containing an indicator and frozen, the indicator being to react with a component generated from a biological sample; a heater configured to heat the first solution in the first container in a support state where the support unit supports the first container; a first addition unit configured to, in the support state, add a second solution containing a stimulant to the first solution in the first container, the stimulant being to activate a function of the biological sample; a second addition unit configured to, in the support state, add the biological sample to the first solution in the first container; a first adjustment unit configured to, in the support state, adjust an addition position of the second solution with respect to a liquid surface of the first solution in the first container; and an optical unit configured to, in the support state, perform irradiation of a mixture of the first solution thawed, and the second solution and the biological sample added to the first solution with excitation light and detection of detection light including fluorescence generated in the mixture by irradiation with the excitation light, in which the first addition unit includes a first nozzle to which a second container accommodating the second solution is attached, and the first nozzle is configured to discharge the second solution from the second container to the first solution, and the first adjustment unit moves the first nozzle along a height direction intersecting the liquid surface and thereby adjusts an addition position of the second solution with respect to the liquid surface in the height direction”.

A measurement device according to the present disclosure is [2] “A measurement device used for measurement of a characteristic of a biological sample, the measurement device including: a support unit for supporting a first container configured to accommodate therein a frozen first solution for diluting the biological sample; a heater configured to heat the first solution in the first container in a support state where the support unit supports the first container; a first addition unit configured to, in the support state, add a second solution containing a stimulant to the first solution in the first container, the stimulant being to activate a function of the biological sample; a second addition unit configured to, in the support state, add the biological sample and an indicator being to react with a component generated from the biological sample to the first solution in the first container; a first adjustment unit configured to, in the support state, adjust an addition position of the second solution with respect to a liquid surface of the first solution in the first container; and an optical unit configured to, in the support state, perform irradiation of a mixture of the first solution thawed, and the second solution, the biological sample, and the indicator added to the first solution with excitation light and detection of detection light including fluorescence generated in the mixture by irradiation with the excitation light, in which the first addition unit includes a first nozzle to which a second container accommodating the second solution is attached, and the first nozzle is configured to discharge the second solution from the second container to the first solution, and the first adjustment unit moves the first nozzle along a height direction intersecting the liquid surface and thereby adjusts an addition position of the second solution with respect to the liquid surface in the height direction”.

A measurement method according to the present disclosure is [9] “A measurement method for measuring a characteristic of a biological sample by using a measurement device, the measurement method including: a first step of causing a support unit of the measurement device to support a first container accommodating therein a first solution containing an indicator and frozen, the indicator being to react with a component generated from a biological sample; a second step of, after the first step, using a heater of the measurement device to thaw the first solution in the first container; a third step of attaching, to a first nozzle of the measurement device, a second container accommodating therein a second solution containing a stimulant to activate a function of the biological sample; a fourth step of, after the second step and the third step, adjusting an addition position of the second solution with respect to a liquid surface of the first solution in the first container; a fifth step of, after the fourth step, adding the second solution to the first solution in the first container; a sixth step of, after the second step, adding the biological sample to the first solution in the first container; and a seventh step of, after the fifth step and the sixth step, using an optical unit of the measurement device to perform irradiation of a mixture of the first solution, and the biological sample and the second solution added to the first solution with excitation light and detection of detection light including fluorescence generated in the mixture by irradiation with the excitation light, in which, in the fourth step, a first adjustment unit of the measurement device moves the first nozzle along a height direction intersecting a liquid surface of the first solution, and thereby adjusts an addition position of the second solution with respect to the liquid surface in the height direction”.

A measurement method according to the present disclosure is [10] “A measurement method for measuring a characteristic of a biological sample by using a measurement device, the measurement method including: a first step of causing a support unit of the measurement device to support a first container accommodating therein a frozen first solution for diluting the biological sample; a second step of, after the first step, using a heater of the measurement device to thaw the first solution in the first container; a third step of attaching, to a first nozzle of the measurement device, a second container accommodating therein a second solution containing a stimulant to activate a function of the biological sample; a fourth step of, after the second step and the third step, adjusting an addition position of the second solution with respect to a liquid surface of the first solution in the first container; a fifth step of, after the fourth step, adding the second solution to the first solution in the first container; a sixth step of, after the second step, adding the biological sample and an indicator being to react with a component generated from the biological sample to the first solution in the first container; and a seventh step of, after the fifth step and the sixth step, using an optical unit of the measurement device to perform irradiation of a mixture of the first solution, and the biological sample, the indicator, and the second solution added to the first solution with excitation light and detection of detection light including fluorescence generated in the mixture by irradiation with the excitation light, in which, in the fourth step, a first adjustment unit of the measurement device moves the first nozzle along a height direction intersecting a liquid surface of the first solution, and thereby adjusts an addition position of the second solution with respect to the liquid surface in the height direction”.

In the measurement device and the measurement method, a first container accommodating therein a first solution that is frozen can be supported by a support unit, and in this state the first solution can be heated by a heater. Thereby, after the first solution is thawed in the vicinity of the subject (in the measurement device), a biological sample collected from the subject can be added to the first solution. Thus, not only is the quality of the first solution maintained, but also operations such as preparation of the first solution and introduction of the first solution into the first container become unnecessary in the vicinity of the subject. Thus, by the measurement device and the measurement method, the quality of the first solution is maintained, and an operation for measuring characteristics of the biological sample is simplified. Further, in the measurement device and the measurement method, the first adjustment unit moves, along the height direction intersecting the liquid surface of the first solution, the first nozzle to which the second container is attached, and thereby adjusts the addition position of the second solution with respect to the liquid surface of the first solution in the height direction. Thereby, the second solution can be added to the first solution at an appropriate addition position with respect to the liquid surface of the first solution.

Here, the findings by the present inventor have revealed that, when adding the second solution to the first solution, if the outlet for the second solution in the second container is far from the liquid surface of the first solution, there is a case where the second solution cannot be completely discharged from the second container, and the amount of the second solution that is discharged from the outlet of the second container and is scattered to the inner wall surface of the first container is increased. On the other hand, if the second solution is discharged from the second container in a state where the outlet for the second solution in the second container is positioned in the first solution, the liquid surface of the first solution is greatly disturbed, and consequently disturbance in the detection signal of detection light in the optical unit is increased. Therefore, there is a demand for suppression of remaining of the second solution, which can have high concentration and the amount of which can be small, in the second container and disturbance in the detection signal.

Thus, the measurement device according to the present disclosure may be [3] “The measurement device according to the above [1] or [2], in which, when adding the second solution to the first solution, the first adjustment unit moves the first nozzle along the height direction such that a distance from the liquid surface in the height direction of an outlet for the second solution in the second container is 1 mm or less in a range in which the outlet is not in contact with the liquid surface”.

Further, the measurement method according to the present disclosure may be [11] “The measurement method according to the above [9] or [10], in which, in the fourth step, when adding the second solution to the first solution, the first adjustment unit of the measurement device moves the first nozzle along the height direction such that a distance from the liquid surface in the height direction of an outlet for the second solution in the second container is 1 mm or less in a range in which the outlet is not in contact with the liquid surface”.

In this case, the distance from the liquid surface of the first solution of the outlet for the second solution in the second container is adjusted to be 1 mm or less in a range in which the outlet is not in contact with the liquid surface of the first solution. When the second solution is thus discharged from the second container at a distance of 1 mm or less from the liquid surface of the first solution, remaining of the second solution in the second container can be suppressed. Further, an increase in the amount of the second solution that is discharged from the second container and is scattered to the inner wall surface of the first container can be suppressed. Further, by preventing the outlet for the second solution in the second container from coming into contact with the first solution, great disturbance of the liquid surface of the first solution due to addition of the second solution can be suppressed, and disturbance in the detection signal can be suppressed.

As described above, in the present disclosure, the first solution is thawed in the measurement device, and a mixture containing the thawed first solution is subjected to optical measurement. In such a case, dew condensation may occur on the surface of the first container during thawing of the first solution, and the dew condensation may influence optical measurement (for example, cause scattering of excitation light and detection light). The influence of dew condensation lasts a long period of time, if measurements are performed only after the condensation has cleared, it becomes time-consuming. Therefore, there is a demand for rapid reduction of the influence of dew condensation on the surface of the first container occurring during thawing of the first solution and shortening of measurement time.

Thus, the measurement device according to the present disclosure may be [4] “The measurement device according to any one of the above [1] to [3], including a blower configured to, in the support state, supply air to the first container, in which the first container includes a light transmission portion configured to transmit the excitation light and the detection light, and the blower supplies the air to a surface of the light transmission portion in the support state”.

Further, the measurement method according to the present disclosure may be [12] “The measurement method according to any one of the above [9] to [11], in which, in the second step, when thawing the first solution, a blower of the measurement device is used to supply air to a surface of a light transmission portion configured to transmit the excitation light and the detection light in the first container”.

In this case, by using a blower to supply air to the surface of the transmission portion for excitation light and detection light in the first container, the occurrence of dew condensation on the first container during thawing of the first solution can be suppressed, and dew condensation occurring on the first container can be quickly removed. Therefore, the influence of dew condensation on the surface of the light transmission portion in the first container can be quickly reduced, and measurement time can be shortened.

The measurement device according to the present disclosure may be [5] “The measurement device according to any one of the above [1] to [4], in which the optical unit includes: an optical filter configured to selectively transmit, out of the detection light from the mixture, the detection light incident at angles of incidence equal to or less than a predetermined angle; a lens configured to condense the detection light transmitted through the optical filter; and a detector configured to detect the detection light condensed by the lens”. In this case, components included in detection light that are influenced by scattering can be removed by the optical filter. Further, even when the amount of detection light is reduced due to some components being removed by the optical filter, the influence of the reduction in the amount of light on the detection result can be suppressed by condensing the detection light with the lens in a stage subsequent to the optical filter.

The measurement device according to the present disclosure may be [6] “The measurement device according to any one of the above [1] to [5], including a second adjustment unit configured to, in the support state, adjust an addition position of the biological sample with respect to the first solution in the first container, in which the second addition unit includes a second nozzle to which a third container accommodating the biological sample is attached, and the second nozzle configured to discharge the biological sample from the third container to the first container, and the second adjustment unit moves the second nozzle along the height direction and thereby adjusts the addition position of the biological sample in the height direction”.

Further, the measurement method according to the present disclosure may be [13] “The measurement method according to any one of the above [9] to [12], including: an eighth step of, before the sixth step, attaching a third container accommodating therein the biological sample to a second nozzle of the measurement device; and a ninth step of, after the eighth step and before the sixth step, adjusting an addition position of the biological sample with respect to the first solution in the first container, in which, in the ninth step, a second adjustment unit of the measurement device moves the second nozzle along the height direction, and thereby adjusts an addition position of the biological sample with respect to the first solution in the height direction”.

In this case, the biological sample can be added to the first solution at an appropriate addition position with respect to the first solution.

The measurement device according to the present disclosure may be [7] “The measurement device according to the above [6], in which the second adjustment unit moves the second nozzle along the height direction such that an outlet for the biological sample in the third container is located in the first solution”.

The measurement method according to the present disclosure may be [14] “The measurement method according to the above [13], in which, in the ninth step, the second adjustment unit of the measurement device moves the second nozzle along the height direction such that an outlet for the biological sample in the third container is located in the first solution”.

In this case, the biological sample can be discharged from the third container in the interior of the first solution.

The measurement device according to the present disclosure may be [8] “The measurement device according to the above [7], in which, when adding the biological sample to the first solution, the second addition unit repeatedly performs discharge of the biological sample from the third container by means of the second nozzle and introduction of the first solution into the third container by means of the second nozzle”.

The measurement method according to the present disclosure may be [15] “The measurement method according to the above [14], in which, in the sixth step, when adding the biological sample to the first solution, discharge of the biological sample from the third container by means of the second nozzle and introduction of the first solution into the third container by means of the second nozzle are repeatedly performed”.

In this case, by performing pipetting at the third container by means of the second nozzle, the biological sample in the third container can be reliably added to the first solution.

According to the present disclosure, a measurement device and a measurement method that enable simple operations can be provided.

### BRIEF DESCRIPTION OF THE DRAWINGS

FIG. 1 is a schematic front view showing a measurement device according to the present embodiment;

FIG. 2 is a schematic side view of the measurement device shown in FIG. 1;

FIG. 3 is an enlarged schematic front view of a metering unit shown in FIGS. 1 and 2;

FIG. 4 is a schematic diagram showing a configuration of an optical unit shown in FIG. 2;

FIG. 5 is a schematic front view showing a manner of adjusting the addition position of a second solution;

FIG. 6 is a flowchart showing steps corresponding to a making-ready stage of a measurement method according to the present embodiment; and

FIG. 7 is a flowchart showing steps of the measurement method according to the present embodiment.

### DETAILED DESCRIPTION

Hereinbelow, embodiments of the present disclosure are described in detail with reference to the drawings. In the drawings, the same or corresponding portions are denoted by the same reference signs, and a repeated description may be omitted.

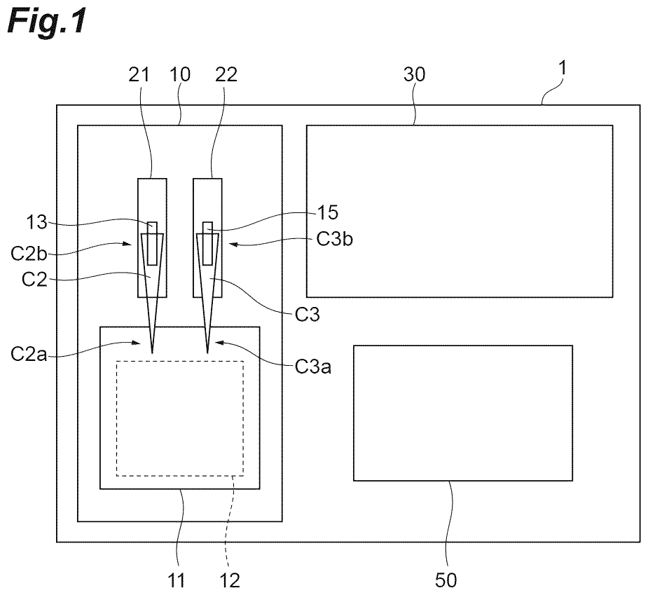

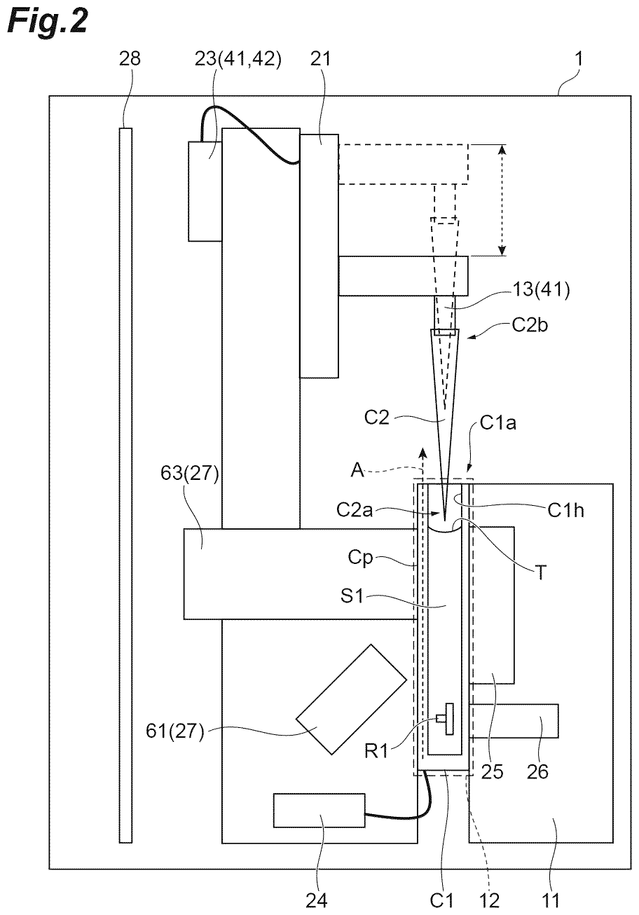

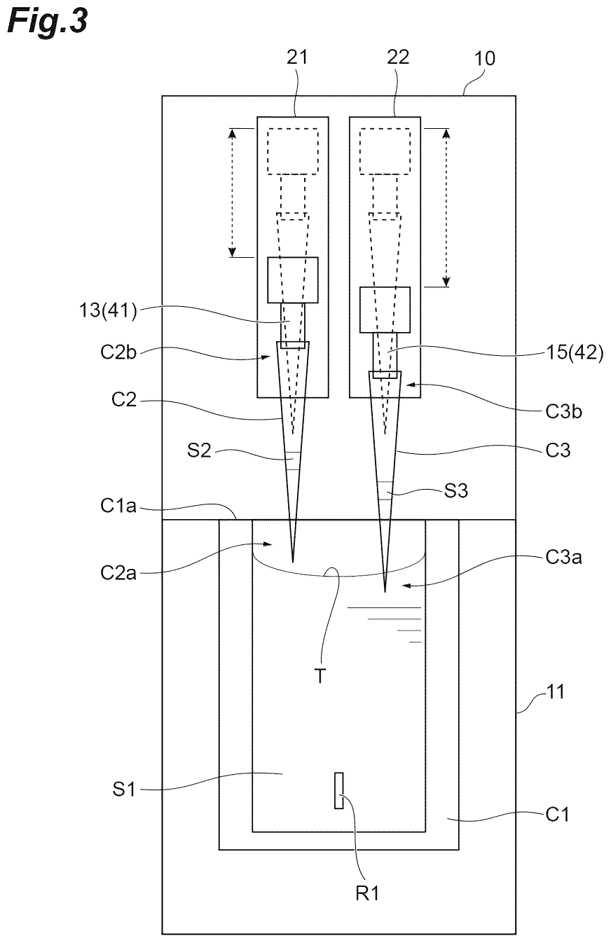

FIG. 1 is a schematic front view showing a measurement device according to the present embodiment. FIG. 2 is a schematic side view of the measurement device shown in FIG. 1. FIG. 3 is an enlarged schematic front view of a metering unit shown in FIGS. 1 and 2. A measurement device 1 shown in FIGS. 1 to 3 is used for measurement of characteristics of a biological sample. The biological sample is, for example, blood (for example, whole blood) of a subject (a living body), or the like. The subject is, for example, a human body or the like. In the present embodiment, the subject is a medical examinee. The biological sample contains, for example, a white blood cell. The characteristics of the biological sample are, for example, the activity of a white blood cell. In the present embodiment, the characteristics of the biological sample are the activity of a neutrophil.

The neutrophil is a type of white blood cell. A main role of the neutrophil is to prevent infection by phagocytosing and sterilizing bacteria and fungi that have entered the living body. The neutrophil wraps a bacterium or the like with a neutrophil plasma membrane, and thereby takes the bacterium or the like into a neutrophil. Thereby, a phagosome is formed. When the phagosome fuses with granules, the granule contents are released into the phagosome. Reactive Oxygen Species (superoxide, hydrogen peroxide) is generated by an NADPH oxidase system formed in a cell membrane (the membrane of the phagosome), and the reactive oxygen species sterilizes the bacterium or the like. Further, hypochlorous acid (HOCl) (or a halogen equivalent thereof) is produced from hydrogen peroxide (H₂O₂) and a chloride ion (Cl⁻) by enzyme reaction of myeloperoxidase (EC number: 1.11.2.2) contained in the granule contents, and the hypochlorous acid sterilizes the bacterium or the like. Accordingly, myeloperoxidase activity or superoxide production activity is used as an index for evaluating the activity of the neutrophil. The measurement device 1 of the present embodiment is used to measure myeloperoxidase activity or superoxide production activity.

The measurement device 1 includes a metering unit 10, a display unit 30, and an output unit 50. The metering unit 10 measures characteristics of a biological sample as described above. The display unit 30 is, for example, a display, and can display measurement results at the metering unit 10. Further, the display unit 30 is, for example, a touch panel, and can be also an input acceptance unit that accepts input from the user. The output unit 50 is, for example, a printer, and can output measurement results at the metering unit 10 by printing. That is, in the measurement device 1, the display unit 30 and the output unit 50 constitute an input/output unit for accepting input of information (for example, a sample name, measurement conditions, etc.) regarding measurement at the metering unit 10 and outputting measurement results at the metering unit 10.

The metering unit 10 has a structure for supporting a plurality of containers that hold a biological sample and various solutions. The containers supported by the metering unit 10 are a first container C1, a second container C2, and a third container C3. The first container C1 accommodates a first solution S1 therein. The first solution S1 is a mixture of a plurality of reagents. The first solution S1 contains, as a plurality of reagents, at least one of a physiological saline solution and a buffer solution, and a fluorescence indicator, etc., for example. The first solution S1 has a function of diluting the biological sample. The fluorescence indicator reacts with a component (HOCl) generated from the biological sample. The fluorescence indicator is, for example, aminophenyl fluorescein (APF) or the like. A commercially available product may be used as the fluorescence indicator. There may be a case where the first solution S1 does not contain a fluorescence indicator. Hereinafter, the case where the first solution S1 does not contain the fluorescence indicator will be referred to as “first modified example.” In addition, the fluorescence indicator may react with components generated from the biological sample, such as reactive oxygen species (for example, superoxide, nitric oxide, and the like). The fluorescence indicator may be, for example, DCFH-DA, BES-So, or DAF-2, and the like.

The first solution S1 is accommodated in the first container C1 in a frozen state. The first solution S1 may be entirely frozen, or may be partially frozen. The temperature of the first solution S1 is equal to or lower than the freezing point of any reagent contained in the first solution S1. That is, the temperature of the first solution S1 may be the same as the freezing point of any reagent contained in the first solution S1, or may be lower than the freezing point of any reagent contained in the first solution S1.

In the present embodiment, the temperature of the first solution S1 is equal to or lower than the freezing point of a reagent having the largest volume among the plurality of reagents contained in the first solution S1. The temperature of the first solution S1 may be equal to or lower than the freezing point of a reagent having the lowest freezing point among the plurality of reagents contained in the first solution S1. In the present embodiment, the temperature of the first solution S1 is equal to or lower than the freezing point of the buffer solution contained in the first solution S1. The temperature of the first solution S1 may be equal to or lower than the freezing point of the fluorescence indicator contained in the first solution S1. In the present embodiment, the temperature of the entire first container C1 is equal to or lower than the freezing point of any reagent contained in the first solution S1. The temperature of the first solution S1 is, for example, about −20° C. In the present embodiment, the “temperature” refers to temperature under atmospheric pressure.

The first container C1 is a box body formed in a rectangular plate shape and having an opening C1h at one end C1a. At least part of the walls forming the first container C1 have a light transmission portion Cp that transmits excitation light L1 and detection light L2 described later. Herein, a light transmission portion Cp is provided on one wall forming the opening C1h among the walls of the first container C1. In the interior (the vicinity of the bottom) of the first container C1, a rotor R1 is provided in a position immersed in the first solution S1. The first container C1, the first solution S1, and the rotor R1 are configured as one measurement kit. Therefore, the first container C1, the first solution S1, and the rotor R1 are supported by the metering unit 10 integrally as a measurement kit.

The second container C2 accommodates a second solution S2 therein. The second solution S2 is a mixture of a plurality of reagents. The second solution S2 contains, as a plurality of reagents, a stimulant, an organic solvent, etc., for example. The stimulant is a reagent for activating a function of the biological sample. The stimulant stimulates, for example, a neutrophil of the biological sample in a pseudo manner. When a neutrophil is stimulated in a pseudo manner, innate immune response (biological defense response) of the neutrophil is triggered. The stimulant is, for example, fMLP (N-formyl-L-methionyl-L-leucyl-phenylalanine), PMA (4β-phorbol-12-myristate-13-acetate), or the like. The organic solvent is a reagent for dissolving a stimulant in a powder state. The organic solvent is, for example, DMSO (dimethyl sulfoxide) or the like.

The second solution S2 is frozen in a state of being accommodated in the second container C2. The second solution S2 may be entirely frozen, or may be partially frozen. The temperature of the second solution S2 is equal to or lower than the freezing point of any reagent contained in the second solution S2. That is, the temperature of the second solution S2 may be the same as the freezing point of any reagent contained in the second solution S2, or may be lower than the freezing point of any reagent contained in the second solution S2. In the present embodiment, the temperature of the second solution S2 is equal to or lower than the freezing point of a reagent having the largest volume among the plurality of reagents contained in the second solution S2. The temperature of the second solution S2 may be equal to or lower than the freezing point of a reagent having the lowest freezing point among the plurality of reagents contained in the second solution S2. In the present embodiment, the temperature of the second solution S2 is equal to or lower than the freezing point of the organic solvent contained in the second solution S2.

In the present embodiment, the temperature of the entire second container C2 is equal to or lower than the freezing point of any reagent contained in the second solution S2. The temperature of the second solution S2 is lower than the temperature of the first solution S1. The temperature of the second solution S2 is, for example, −40° C. or lower. In the present embodiment, the temperature of the second solution S2 is −80° C. The temperature of the second solution S2 may be −20° C. or lower.

In the present embodiment, the second solution S2 does not contain a buffer solution. Therefore, deterioration of the stimulant caused by a buffer solution is suppressed. Specifically, as the period until the freezing of the second solution S2 becomes longer, the stimulant diluted with a buffer solution tends to deteriorate. In the case where the second solution S2 does not contain a buffer solution, even if the period until the freezing of the second solution S2 is relatively long, deterioration of the stimulant is suppressed.

The second solution S2 may further contain a buffer solution (buffer). The buffer solution is a reagent for diluting the organic solvent. Since the organic solvent is diluted with a buffer solution in the second solution S2, even if the second solution S2 is directly applied to the biological sample, damage to the biological sample due to the organic solvent is suppressed. As each of the stimulant, the organic solvent, and the buffer solution, for example, a commercially available product may be used. However, if the organic solvent is diluted with a buffer solution, the stimulant dissolved in the organic solvent may be likely to deteriorate. In this regard, by setting the temperature of the second solution S2 to be lower than the temperature of the first solution S1 as described above (setting the temperature to extremely low temperature), even if the organic solvent is diluted with a buffer solution in order to directly apply the second solution S2 to the biological sample, the second solution S2 can be preserved while a state where deterioration of the stimulant dissolved in the organic solvent is suppressed is maintained.

The second container C2 is formed in, for example, a conical shape having openings at one end C2a and the other end C2b. The one end C2a of the second container C2 corresponds to, for example, the apex of the cone, and is a portion serving as an outlet for the second solution S2. The other end C2b of the second container C2 corresponds to, for example, the bottom surface of the cone, is attached to a first nozzle 13 described later, and is a portion serving as an inlet for air from the first nozzle 13. The second container C2 is, for example, a pipette tip. The second container C2 and the second solution S2 are supported by the metering unit 10 integrally as a measurement kit.

The third container C3 accommodates a biological sample S3 therein. The biological sample S3 is collected from a subject, and is accommodated in the third container C3. The third container C3 is formed in a conical shape having openings at one end C3a and the other end C3b. The one end C3a of the third container C3 corresponds to, for example, the apex of the cone, and is a portion serving as an outlet (and an inlet) for the biological sample S3, etc. The other end C3b of the third container C3 corresponds to, for example, the bottom surface of the cone, is attached to a second nozzle 15 described later, and is a portion serving as an inlet (and an outlet) for air from the second nozzle 15. In the first modified example, the third container C3 may further accommodate the fluorescence indicator in addition to the biological sample S3. Alternatively, in the first modified example, a plurality of third containers C3 may be prepared, and then the biological sample S3 may be contained in one of the third containers C3 and the fluorescence indicator may be contained in the other third container C3. Further, in the first modified example, the fluorescence indicator may be cooled or frozen in a state where contained in the third container C3, as described later.

As described later, pipetting can be performed at the third container C3. In this case, discharge and introduction of liquid on the third container C3 are repeated. When discharging liquid from the third container C3, the other end C3b serves as an inlet for air from the second nozzle 15, and the one end C3a serves as an outlet for the liquid. When introducing liquid into the third container C3, the other end C3b serves as an outlet for air to the second nozzle 15, and the one end C3a serves as an inlet for the liquid. The third container C3 is, for example, a pipette tip. In the first modified example, at least one of the one end C3a and the other end C3b may serve as an inlet for introducing a liquid such as the fluorescence indicator into the third container C3.

In the measurement device 1, the metering unit 10 supports (holds) the first container C1, the second container C2, and the third container C3 described above. More specifically, the metering unit 10 includes a holder 11 for the first container C1, a first nozzle 13, and a second nozzle 15. The holder 11 is provided with an insertion unit 12 (a support unit) for the first container C1. The insertion unit 12 holds the first container C1, and thereby supports the first container C1 at a fixed position in the metering unit 10. The insertion unit 12 supports the first container C1 in an attitude in which the opening C1h at the one end C1a of the first container C1 faces (opens) vertically upward and the light transmission portion Cp of the first container C1 faces an optical unit 27 described later.

The first nozzle 13 is located vertically above the insertion unit 12. The first nozzle 13 is placed to face the opening C1h of the first container C1 in a state where the insertion unit 12 supports the first container C1 (hereinafter, referred to as a “support state”). The second container C2 is attached to the first nozzle 13. The second container C2 is attached to the first nozzle 13 by the tip of the first nozzle 13 being inserted (for example, fitted) into the other end C2b. In other words, the first nozzle 13 supports (holds) the second container C2 in the metering unit 10 such that the one end C2a of the second container C2 faces the opening C1h side of the first container C1.

The second nozzle 15 is located above the insertion unit 12. In the support state, the second nozzle 15 is placed to face the opening C1h of the first container C1. The first nozzle 13 and the second nozzle 15 are, for example, arranged along the horizontal direction. The third container C3 is attached to the second nozzle 15. The third container C3 is attached to the second nozzle 15 by the tip of the second nozzle 15 being inserted (for example, fitted) into the other end C3b. In other words, the second nozzle 15 supports (holds) the third container C3 in the metering unit 10 such that the one end C3a of the third container C3 faces the opening C1h side of the first container C1.

The metering unit 10 is provided with a metering unit lid (not illustrated). Thereby, the metering unit 10 is configured such that a state of being opened to the outside (an opened state of the lid) and a state of being shielded (light-shielded) from the outside (a closed state of the lid) can be switched by opening and closing of the metering lid.

The measurement device 1 further includes a first adjustment unit 21, a second adjustment unit 22, blowers 23 and 24, a heater 25, a motor 26, an optical unit 27, and a circuit board 28. The first adjustment unit 21 is a member for, in the support state, adjusting the addition position of the second solution S2 with respect to the liquid surface T of the first solution S1 in the first container C1. The first adjustment unit 21 is, for example, an electric slider, and the first nozzle 13 is attached thereto. The first adjustment unit 21 moves the first nozzle 13 along the height direction (herein, the vertical up-down direction) intersecting the liquid surface T of the first solution S1 to adjust the position in the height direction of the one end C2a of the second container C2 (that is, the outlet for the second solution S2) attached to the first nozzle 13, and thus adjusts the addition position of the second solution S2 with respect to the liquid surface T in the height direction.

The second adjustment unit 22 is a member for, in the support state, adjusting the addition position of the biological sample S3 with respect to the first solution S1 in the first container C1. In the first modified example, the second adjustment unit 22 may be a member for, in the support state, adjusting the addition position of the biological sample S3 and the fluorescence indicator with respect to the first solution S1 in the first container C1. The second adjustment unit 22 is, for example, an electric slider, and the second nozzle 15 is attached thereto. The second adjustment unit 22 moves the second nozzle 15 along the height direction to adjust the position in the height direction of the one end C3a of the third container C3 (that is, the outlet for the biological sample S3) attached to the second nozzle 15, and thus adjusts the addition position of the biological sample S3 in the height direction. In the first modified example, the second adjustment unit 22 may move the second nozzle 15 along the height direction to adjust the position in the height direction of the one end C3a of the third container C3 (that is, the outlet for the biological sample S3 and the fluorescence indicator) attached to the second nozzle 15, and thus may adjust the addition position of the biological sample S3 and the fluorescence indicator in the height direction.

In the present embodiment, the measurement device 1 separately includes the first adjustment unit 21 that moves the first nozzle 13 along the height direction and the second adjustment unit 22 that moves the second nozzle 15 along the height direction, and can operate these units independently of each other. However, the measurement device 1 may unify the first adjustment unit 21 and the second adjustment unit 22, and may include one adjustment unit that collectively moves the first nozzle 13 and the second nozzle 15 along the height direction.

The blower 23 is connected to the first nozzle 13 and the second nozzle 15, and is a member for ejecting air to the first nozzle 13 and the second nozzle 15 or sucking air from the second nozzle 15. By ejecting air to the first nozzle 13, the blower 23 introduces air into the second container C2 through the other end C2b of the second container C2 attached to the first nozzle 13, and causes the second solution S2 to be discharged through the one end C2a of the second container C2. That is, in the support state, the first nozzle 13 and the blower 23 constitute a first addition unit 41 for adding the second solution S2 to the first solution S1 in the first container C1.

Further, by ejecting air to the second nozzle 15, the blower 23 introduces air into the third container C3 through the other end C3b of the third container C3 attached to the second nozzle 15, and causes the biological sample S3 to be discharged through the one end C3a of the third container C3. Further, by sucking air from the second nozzle 15, the blower 23 causes air in the third container C3 to be discharged through the other end C3b of the third container C3, and causes the first solution S1 to be introduced into the third container C3 through the one end C3a of the third container C3. Thus, in the support state, the second nozzle 15 and the blower 23 constitute a second addition unit 42 for adding the biological sample S3 to the first solution S1 in the first container C1. In the case where pipetting at the third container C3 is performed as described later, the liquid first discharged from the third container C3 is the biological sample S3, and the liquid introduced into the third container C3 is a liquid in a state where the biological sample S3 is added to the first solution S1. Further, the liquids discharged from the third container C3 for the second and subsequent times are mixtures of the biological sample S3 and the first solution S1. In the first modified example, by ejecting air to the second nozzle 15, the blower 23 may introduce air into the third container C3 through the other end C3b of the third container C3 attached to the second nozzle 15, and may cause the biological sample S3 and the fluorescence indicator to be discharged through the one end C3a of the third container C3. In the first modified example, in the support state, the second nozzle 15 and the blower 23 may constitute a second addition unit 42 for adding the biological sample S3 and the fluorescence indicator to the first solution S1 in the first container C1. In the case where pipetting at the third container C3 is performed, the liquid first discharged from the third container C3 may be the biological sample S3 and the fluorescence indicator, and the liquid introduced into the third container C3 may be a liquid in a state where the biological sample S3 and the fluorescence indicator are added to the first solution S1. Further, the liquids discharged from the third container C3 for the second and subsequent times may be mixtures of the biological sample S3, the fluorescence indicator, and the first solution S1. In the first modified example, when a plurality of third containers C3 are used, a plurality of second addition units 42 may be configured by the respective second nozzles 15 to which the respective third containers C3 are attached, together with the blower 23. For example, when the biological sample S3 is accommodated in one third container C3 and the fluorescence indicator is accommodated in the other third container C3, two second addition units 42 may be configured respectively by the second nozzle 15 to which the one third container C3 is attached and the blower 23, and by the second nozzle 15 to which the other third container C3 is attached and the blower 23.

Although in the present embodiment one blower 23 is provided for the first nozzle 13 and the second nozzle 15, separate blowers that operate independently of each other may be provided for the first nozzle 13 and the second nozzle 15.

The blower 24 is a member for, in the support state, supplying air to the first container C1. In the support state, the blower 24 supplies air A to at least the surface of the light transmission portion Cp of the first container C1. As an example, the blower 24 can form an air flow flowing vertically upward on the wall surface including the surface of the light transmission portion Cp in the first container C1. Thus, the blower 24 can forcibly ventilate a space in the vicinity of the light transmission portion Cp. The blower 24 may be unified with the blower 23.

The heater 25 is provided in the holder 11 of the metering unit 10. In the present embodiment, in the support state, the heater 25 is provided to face a wall of the first container C1 on the opposite side to the wall on which the light transmission portion Cp is provided. The heater 25 is a member for, in the support state, heating the first solution S1 in the first container C1. The heater 25 can thaw the first solution S1 in the first container C1 by heating. Further, after the first solution S1 in the first container C1 is thawed, the heater 25 can heat the first solution S1 so that the temperature of the first solution S1 reaches a suitable temperature (optimum temperature) for the biological sample S3, and can heat the first solution S1 so that the first solution S1 is maintained at the suitable temperature.

The motor 26 is provided in the holder 11 of the metering unit 10. In the present embodiment, in the support state, the motor 26 is provided to face a wall of the first container C1 on the opposite side to the wall on which the light transmission portion Cp is provided. In the support state, the motor 26 is placed to face the rotor R1 in the first container C1. The motor 26 forms, for example, a magnetic stirrer, and stirs the first solution S1 by rotating the rotor R1.

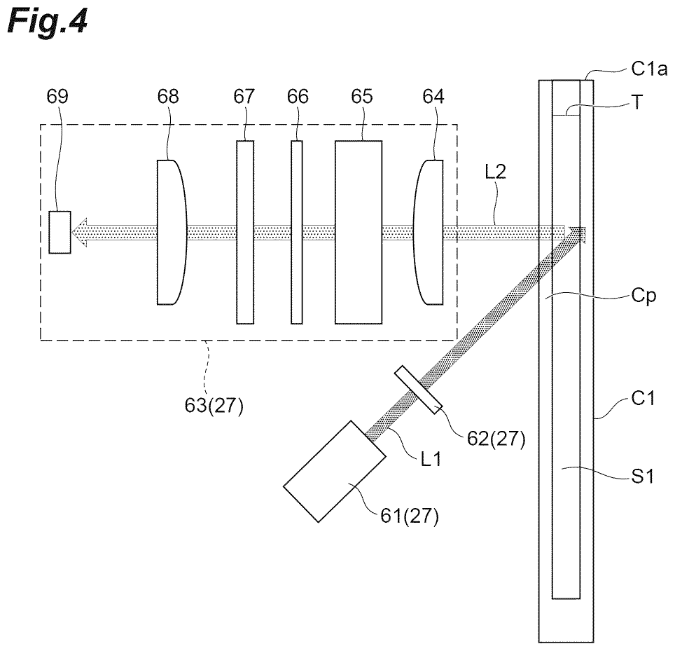

FIG. 4 is a schematic diagram showing a configuration of the optical unit shown in FIG. 2. As shown in FIGS. 2 and 4, in the support state, the optical unit 27 is provided to face the light transmission portion Cp in the first container C1. The optical unit 27 is a member for, in the support state, performing irradiation of a mixture of a thawed first solution S1, and a second solution S2 and a biological sample S3 added to the first solution S1 with excitation light L1 and detection of detection light L2 including fluorescence generated in the mixture by the irradiation with excitation light L1. In the first modified example, the optical unit 27 may be a member for, in the support state, performing irradiation of a mixture of a thawed first solution S1, and a second solution S2, a biological sample S3, and the fluorescence indicator added to the first solution S1 with excitation light L1 and detection of detection light L2 including fluorescence generated in the mixture by the irradiation with excitation light L1.

The optical unit 27 includes a light source 61, an optical filter 62, and a lens barrel unit 63. The light source 61 includes, for example, a light emitting element such as a laser diode or a light emitting diode. In the support state, the light source 61 emits excitation light L1 toward the light transmission portion Cp. The optical filter 62 is provided on the optical path of excitation light L1 emitted from the light source 61. The optical filter 62 transmits a portion of wavelength components of the excitation light L1 emitted from the light source 61. The optical filter 62 is, for example, a short-pass filter that transmits wavelength components of 490 nm or less.

Thus, the optical unit 27 irradiates a mixture in the first container C1 with excitation light L1 via the light transmission portion Cp. Thereby, in the first container C1, a substance generated by a reaction between a component generated from the biological sample S3 and the fluorescence indicator of the first solution S1 is excited; as a result, fluorescence is generated. Therefore, detection light L2 including the fluorescence is emitted from the light transmission portion Cp of the first container C1.

The lens barrel unit 63 includes a lens 64, optical filters 65 to 67, a lens 68, and a detector 69 that are arranged in order on the optical path of detection light L2 emitted via the light transmission portion Cp. The lens 64 and the lens 68 are, for example, plano-convex lenses that are convex on the sides facing each other, and condense, toward the detector 69, detection light L2 emitted from the light transmission portion Cp. The lens 64 and the lens 68 constitute a relay lens system.

The optical filter 65 is a member for selectively transmitting, out of the detection light L2 emitted from the lens 64, detection light L2 incident at angles of incidence equal to or less than a predetermined angle (for example, 10°). The optical filter 65 is, for example, a honeycomb filter. The optical filters 66 and 67 transmit a portion of wavelength components of the detection light L2 from the optical filter 65. The optical filter 66 is, for example, a long-pass filter that transmits wavelength components of 530 nm or more. The optical filter 67 is, for example, a long-pass filter that transmits wavelength components of 515 nm or more. The lens 68 condenses detection light L2 transmitted through the optical filters 65 to 67. The detector 69 detects detection light L2 condensed by the lens 68. The detector 69 includes, for example, a photodiode.

The circuit board 28 is electrically connected to each part of the measurement device 1. In the circuit board 28, a control unit that controls each part of the measurement device 1 may be configured. In this case, the control unit can control, for example, at least one of the movement of the first nozzle 13 by the first adjustment unit 21, the movement of the second nozzle 15 by the second adjustment unit 22, the ejection and suction of air at the blower 23, the supply of air to the first container C1 by the blower 24, and the emission of excitation light L1 by the light source 61.

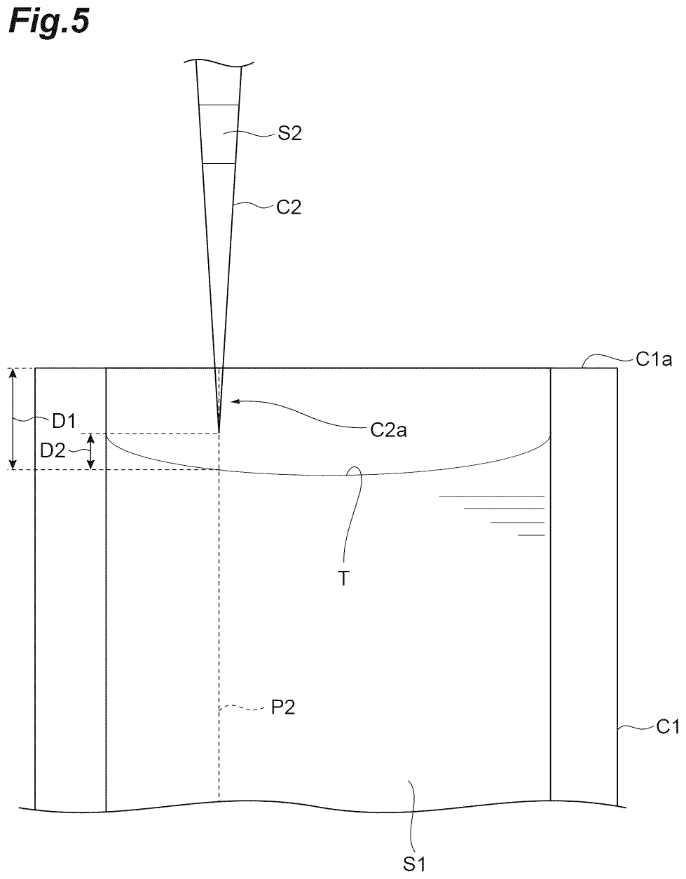

Here, the adjustment of the addition position of the second solution S2 by the first adjustment unit 21 and the adjustment of the addition position of the biological sample S3 by the second adjustment unit 22 will now be described in more detail. FIG. 5 is a schematic front view showing a manner of adjusting the addition position of the second solution. As shown in FIGS. 3 and 5, the first adjustment unit 21 moves the first nozzle 13 along the height direction such that the one end C2a of the second container C2 attached to the first nozzle 13 (that is, the outlet for the second solution S2 and at the same time the addition position) is at an appropriate distance away from the liquid surface T of the first solution S1 accommodated in the first container C1.

The findings by the present inventor have revealed that, when the distance D2 between the one end C2a of the second container C2 and the liquid surface T of the first solution S1 is as large as, for example, 3 mm or more, there is a case where, due to liquid return, the second solution S2 cannot be completely discharged from the second container C2. In contrast, when the distance D2 between the one end C2a of the second container C2 and the liquid surface T of the first solution S1 is set to 1 mm or less, the second solution S2 can be sufficiently discharged from the second container C2. On the other hand, if the one end C2a of the second container C2 is located in the first solution S1, although the second solution S2 can be sufficiently discharged from the second container C2, disturbance of the liquid surface T at the time of adding the second solution S2 is increased.

Thus, when adding the second solution S2 to the first solution S1, the first adjustment unit 21 moves the first nozzle 13 along the height direction such that the distance D2 from the liquid surface T in the height direction of the outlet for the second solution S2 in the second container C2 (the one end C2a) is 1 mm or less in a range in which the one end C2a is not in contact with the liquid surface T. Such processing may be performed by a control unit configured on the circuit board 28 controlling the first adjustment unit 21.

When the one end C2a of the second container C2 is, in the horizontal direction, positioned at a position P2 about ¼ from the end of the liquid surface T as an example and the distance D1 from the one end C1a of the first container C1 to the liquid surface T at the position P2 is about 5 mm, the first adjustment unit 21 can move the first nozzle 13 such that, in the height direction, the one end C2a of the second container C2 is at a position about 4 mm (=distance D1−distance D2) from the one end C1a of the first container C1. The distance D2 may be equal to or less than 0.5 mm (for example, about 0.5 mm). In this case, the first adjustment unit 21 may move the first nozzle 13 such that, in the height direction, the one end C2a of the second container C2 is at a position about 4.5 mm (=distance D1−distance D2) from the one end C1a of the first container C1.

On the other hand, when adding the biological sample S3 to the first solution S1, from a requirement in terms of, for example, performing pipetting, the second adjustment unit 22 moves the second nozzle 15 along the height direction such that the one end C3a of the third container C3 (that is, the outlet for the biological sample S3) is located in the first solution S1. Thus, the second addition unit 42 can suitably perform pipetting by repeatedly performing the discharge of the biological sample S3 from the third container C3 by means of the second nozzle 15 and the introduction of the first solution S1 into the third container C3 by means of the second nozzle 15. Also such processing may be performed by a control unit configured on the circuit board 28 controlling the second adjustment unit 22 and the blower 23. In the first modified example, the second adjustment unit 22 may move the second nozzle 15 along the height direction such that the one end C3a of the third container C3 (that is, the outlet for the biological sample S3 and the fluorescence indicator) is located in the first solution S1. Thus, the second addition unit 42 may suitably perform pipetting by repeatedly performing the discharge of the biological sample S3 and the fluorescence indicator from the third container C3 by means of the second nozzle 15 and the introduction of the first solution S1 into the third container C3 by means of the second nozzle 15.

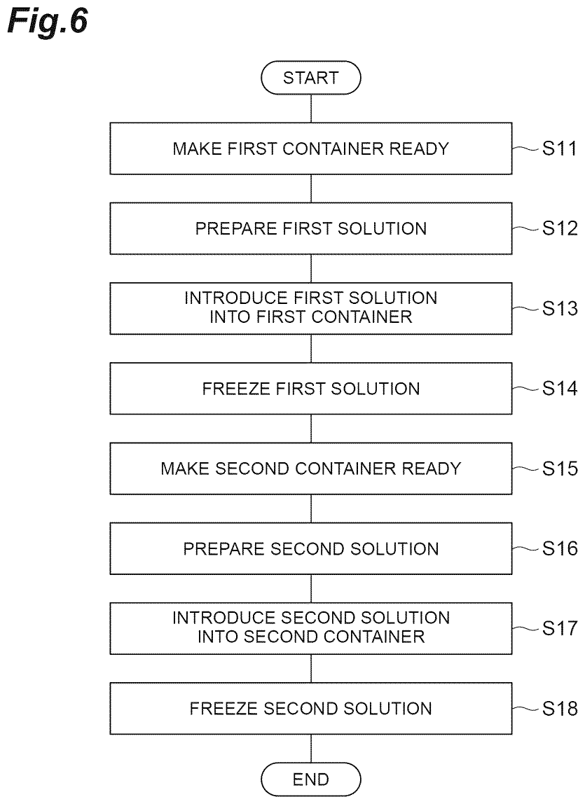

Subsequently, an example of a making-ready step of a measurement method using the measurement device 1 according to the above embodiment is described. FIG. 6 is a flowchart showing steps corresponding to a making-ready stage of a measurement method according to the present embodiment. As shown in FIG. 6, herein, first, a first container C1 accommodating a rotor R1 therein is made ready (step S11). Together with this, a plurality of reagents (at least one of a physiological saline solution and a buffer solution, and a fluorescence indicator, etc.) are mixed outside the first container C1, and thereby a first solution S1 is prepared (step S12). In the first modified example, a first solution S1 which does not contain the fluorescence indicator may be prepared in step S12. Subsequently, the first solution S1 prepared in advance is introduced into the first container C1 (step S13). In the first modified example, the fluorescence indicator may be introduced into the third container C3 in step S13.

Subsequently, the first solution S1 is frozen in a state of being accommodated in the first container C1 (step S14). In the first modified example, the fluorescence indicator may be cooled to approximately −20° C. in a state of being accommodated in the third container C3 in step S14. In step S14, the first container C1 is placed in a cooling space of a cooling facility in a state where the rotor R1 accommodated in the first container C1 is immersed in the first solution S1, and thereby the first solution S1 is frozen. The temperature of the cooling space is, for example, about −20° C. The first container C1 is placed in the cooling space for about two hours, for example. Thereby, the first solution S1 is frozen, and a measurement kit composed of the first container C1, the first solution S1, and the rotor R1 is manufactured. The opening C1h of the first container C1 may be sealed with a sealing member such as a Parafilm or a rubber stopper. In the first modified example, in step S14, a measurement kit including the third container C3 may be manufactured by, for example, cooling the fluorescence indicator in the third container C3 to about −20° C. or by freezing the fluorescence indicator in the third container C3.

On the other hand, a second container C2 is made ready (step S15). Together with this, a plurality of reagents (a stimulant, an organic solvent, etc.) are mixed outside the second container C2, and thereby a second solution S2 is prepared (step S16). Subsequently, the second solution S2 prepared in advance is introduced into the second container C2 (step S17). After that, the second container C2 is placed in a cooling space of a cooling facility, and thereby the second solution S2 is frozen in a state of being accommodated in the second container C2 (step S18). The temperature of the cooling space is, for example, about −80° C. The second container C2 is placed in the cooling space for about one hour, for example. Thereby, the second solution S2 is frozen, and a measurement kit composed of the second container C2 and the second solution S2 is manufactured. As described above, a measurement unit is manufactured with the two measurement kits: the measurement kit including the first container C1 manufactured in step S14, and the measurement kit including the second container C2 manufactured in step S18. On the other hand, in the first modified example, a measurement unit may be manufactured with three measurement kits: the measurement kit including the first container C1 manufactured and the measurement kit including the third container C3 in step S14,, and the measurement kit including the second container C2 manufactured in step S18.

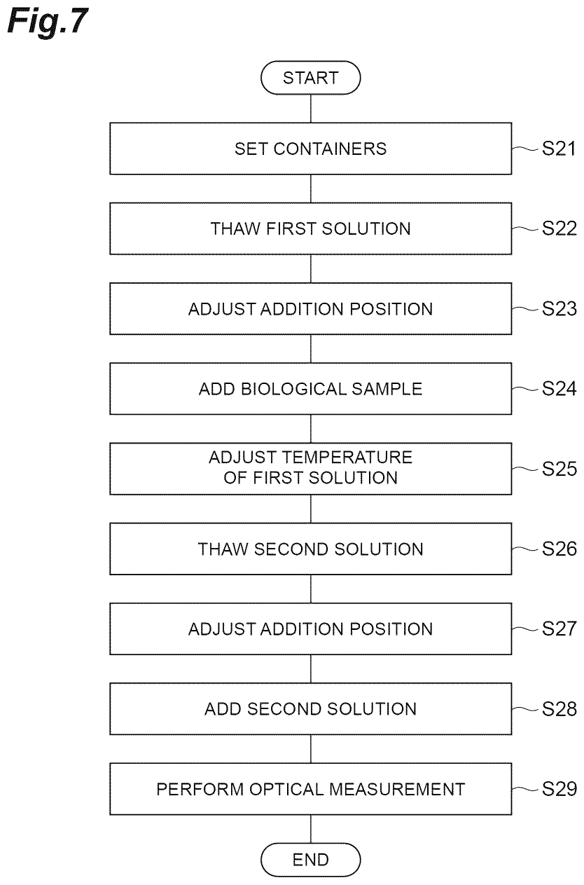

Subsequently, an example of the measurement method using the measurement device 1 according to the above embodiment is described. FIG. 7 is a flowchart showing steps of the measurement method according to the present embodiment. As shown in FIG. 7, herein, first, as making-ready of the measurement device 1, the measurement device 1 is turned on, and then a warm-up operation of the measurement device 1 is performed until the temperature of the insertion unit 12 of the metering unit 10 reaches a suitable temperature (for example, 37° C.) (not illustrated). As an example, the temperature of the insertion unit 12 may be displayed on the display unit 30.

Then, after the warm-up operation of the measurement device 1 is finished, the first container C1, the second container C2, and the third container C3 are readied, and are set in the measurement device 1 (step S21). In step S21, a frozen first solution S1 and a rotor R1 are accommodated in the interior of the first container C1. Further, a frozen second solution S2 is accommodated in the interior of the second container C2. Further, a biological sample S3 collected from a subject is accommodated in the interior of the third container C3. In the first modified example, the biological sample S3 and the fluorescence indicator may be accommodated in the interior of the third container C3. Then, in step S21, the insertion unit 12 of the metering unit 10 is caused to hold the first container C1 and thereby the first container C1 is set in the measurement device 1, the second container C2 is attached to the first nozzle 13 and is thereby set in the measurement device 1, and the third container C3 is attached to the second nozzle 15 and is thereby set in the measurement device 1.

That is, in step S21, the insertion unit 12 of the measurement device 1 is caused to support a first container C1 accommodating therein a first solution S1 that contains an indicator to react with a component generated from a biological sample S3 and that is frozen (a first step). Further, in step S21, a second container C2 accommodating therein a second solution S2 that contains a stimulant to activate a function of the biological sample S3 is attached to the first nozzle 13 of the measurement device 1 (a third step). Further, in step S21, a third container C3 accommodating the biological sample S3 therein is attached to the second nozzle 15 of the measurement device 1 (an eighth step). After that, input of a sample name may be accepted from the user via the display unit 30. In the first modified example, in step S21, the insertion unit 12 of the measurement device 1 may be caused to support the first container C1 accommodating therein the frozen first solution S1 for diluting the biological sample S3 (the first step). Further, in the first modified example, in step S21, the third container C3 accommodating the biological sample S3 and the fluorescence indicator therein may be attached to the second nozzle 15 of the measurement device 1 (the eighth step). In the first modified example, in a case where the fluorescence indicator in the third container C3 is cooled to approximately −20° C., the fluorescent indicator in the third container C3 may be warmed prior to introducing the biological sample S3 into the third container C3, for example by holding the third container C3 at room temperature for a predetermined period of time. In the first modified example, in a case where the fluorescence indicator in the third container C3 is frozen, the fluorescent indicator in the third container C3 may be thawed naturally prior to introducing the biological sample S3 into the third container C3, for example by holding the third container C3 at room temperature for a predetermined period of time.

Subsequently, in the support state, the first solution S1 in the first container C1 is heated by the heater 25, and thereby the first solution S1 in the first container C1 is thawed (step S22, a second step). In step S22, for example, warming of the first solution S1 by the heater 25 is maintained (for example, heating is maintained for about 15 minutes) so that the temperature of the first solution S1 reaches a suitable temperature (optimum temperature) for the biological sample S3. The suitable temperature for the biological sample S3 is, for example, 37° C.

In step S22, heating of the first solution S1 is performed while the rotor R1 is rotated by the motor 26. Specifically, in step S22, the first solution S1 becomes able to flow as a result of being thawed, and then the rotor R1 is rotated. When the rotor R1 rotates, the first solution S1 is stirred in the first container C1; thereby, heat transfer in the first solution S1 is promoted, and the efficiency of heating the first solution S1 is improved. In step S22, the motor 26 may be started before the first solution S1 becomes able to flow. In this case, the rotor R1 rotates at the same time as the first solution S1 becomes able to flow. In step S22, when the temperature of the first solution S1 reaches a suitable temperature, information for notifying the user that the heating of the first solution S1 is completed may be presented to the user (for example, by sounding of a buzzer or displaying of the display unit 30).

Here, in the measurement method according to the present embodiment, when thawing the first solution S1 in step S22, the blower 24 of the measurement device 1 is used to supply air to the surface of the light transmission portion Cp of the first container C1. Thus, by performing forced ventilation between the light transmission portion Cp and the optical unit 27, the occurrence of dew condensation on the surface of the light transmission portion Cp is suppressed, and dew condensation occurring on the surface of the light transmission portion Cp is removed.

In the subsequent step, the addition position of the biological sample S3 with respect to the first solution S1 in the first container C1 is adjusted (step S23, a ninth step). More specifically, in step S23, the second adjustment unit 22 of the measurement device 1 moves, along the height direction, the second nozzle 15 to which the third container C3 is attached, and thereby adjusts the position of the one end C3a of the third container C3, which is the addition position of the biological sample S3 with respect to the first solution S1, in the height direction. In particular, in step S23, as shown in FIG. 3, the second adjustment unit 22 moves the second nozzle 15 along the height direction such that the one end C3a of the third container C3 is located in the first solution S1. In the first modified example, the addition position of the biological sample S3 and the fluorescence indicator with respect to the first solution S1 in the first container C1 may be adjusted in step S23. More specifically, in the first modified example, in step S23, the second adjustment unit 22 of the measurement device 1 may moves, along the height direction, the second nozzle 15 to which the third container C3 is attached, and thereby may adjust the position of the one end C3a of the third container C3, which is the addition position of the biological sample S3 and the fluorescence indicator with respect to the first solution S1, in the height direction.

Subsequently, air is ejected from the blower 23 to the second nozzle 15, thereby air is introduced into the third container C3 through the second nozzle 15 and the biological sample S3 is caused to be discharged through the one end C3a of the third container C3, and thus the biological sample S3 is added to the first solution S1 in the first container C1 (step S24, a sixth step). As described above, the third container C3 accommodating the biological sample S3 is set such that the one end C3a, which is the outlet for the biological sample S3, is located in the first solution S1. Therefore, in step S24, addition of the biological sample S3 is performed in the first solution S1. In the first modified example, in step S24, air may be ejected from the blower 23 to the second nozzle 15, thereby air may be introduced into the third container C3 through the second nozzle 15 and the biological sample S3 and the fluorescence indicator may be caused to be discharged through the one end C3a of the third container C3, and thus the biological sample S3 and the fluorescence indicator may be added to the first solution S1 in the first container C1. In the first modified example, in step S24, the third container C3 accommodating the biological sample S3 and the fluorescence indicator may be set such that the one end C3a, which is the outlet for the biological sample S3 and the fluorescence indicator, are located in the first solution S1. Therefore, in the first modified example, in step S24, addition of the biological sample S3 and the fluorescence indicator may be performed in the first solution S1.

Further, in step S24, when adding the biological sample S3 to the first solution S1, the ejection of air from the blower 23 to the second nozzle 15 and the suction of air from the second nozzle 15 are alternately repeated, and thereby the discharge of the biological sample S3 from the third container C3 by means of the second nozzle 15 and the introduction of the first solution S1 into the third container C3 by means of the second nozzle 15 are repeatedly performed. That is, in step S24, pipetting is performed. At this time, as an example, ejection of 400 ms, suction of 200 ms, ejection of 200 ms, suction of 200 ms, and ejection of 400 ms can be sequentially performed. In the first modified example, in step S24, when adding the biological sample S3 and the fluorescence indicator to the first solution S1, the ejection of air from the blower 23 to the second nozzle 15 and the suction of air from the second nozzle 15 may be alternately repeated, and thereby the discharge of the biological sample S3 and the fluorescence indicator from the third container C3 by means of the second nozzle 15 and the introduction of the first solution S1 into the third container C3 by means of the second nozzle 15 may be repeatedly performed.

After the addition of the biological sample S3 (in the first modified example, of the biological sample S3 and the fluorescence indicator) is completed in step S24, the second adjustment unit 22 can raise the second nozzle 15 to the standby position.

In the subsequent step, the heater 25 is used to adjust the temperature of the first solution S1 (step S25). In step S25, the first solution S1 is heated by the heater 25, and thereby the temperature of the first solution S1 is kept at the above suitable temperature for the biological sample. As an example, in step S25, the first solution S1 can be heated such that the temperature of the first solution S1 is, for example, 36.8° C. to 37.2° C. Further, in step S25, the first solution S1 can be heated while the rotor R1 is rotated by the motor 26. This step S25 may be continuously performed until the measurement of characteristics of the biological sample S3 is finished.

Subsequently, the second solution S2 is thawed (step S26). More specifically, in step S26, in a state where the second container C2 is attached to the first nozzle 13, the second container C2 is allowed to stand for a predetermined period of time (for example, 1 to 2 minutes), and thereby the second solution S2 is naturally thawed. Thus, step S26 can be performed after the second container C2 is attached to the first nozzle 13 and simultaneously while other steps are performed.

Subsequently, the addition position of the second solution S2 with respect to the liquid surface T of the first solution S1 (herein, a mixture of the first solution S1 and the biological sample S3) in the first container C1 is adjusted (step S27, a fourth step). More specifically, in step S27, the first adjustment unit 21 moves the first nozzle 13 along the height direction, and thereby adjusts the position of the one end C2a of the second container C2, which is the addition position with respect to the liquid surface T of the first solution S1, in the height direction. In particular, in step S27, as shown in FIG. 5, the first adjustment unit 21 moves the first nozzle 13 along the height direction such that the distance D2 from the liquid surface T of the one end C2a of the second container C2 (the outlet for the second solution S2) is 1 mm or less in a range in which the one end C2a is not in contact with the liquid surface T.

Subsequently, the second solution S2 is added to the first solution S1 in the first container C1 (step S28, a fifth step). More specifically, in step S28, in a state where the addition position of the second solution S2 is adjusted as described above, the blower 23 ejects air to the first nozzle 13, thereby air is introduced into the second container C2 through the first nozzle 13 and the second solution S2 is caused to be discharged through the one end C2a of the second container C2, and thus the second solution S2 is added to the first solution S1 in the first container C1.

After the addition of the second solution S2 is completed in step S28, the first adjustment unit 21 can raise the first nozzle 13 to the standby position.

Subsequently, the optical unit 27 of the measurement device 1 is used to perform irradiation of the mixture of the first solution S1, and the second solution S2 and the biological sample S3 added to the first solution S1 with excitation light L1 and detection of detection light L2 including fluorescence generated in the mixture by the irradiation with excitation light L1; thereby, characteristics of the biological sample S3 are measured (step S29, a seventh step). In the first modified example, the optical unit 27 of the measurement device 1 may be used to perform irradiation of the mixture of the first solution S1, and the second solution S2, the biological sample S3, and the fluorescence indicator added to the first solution S1 with excitation light L1 and detection of detection light L2 including fluorescence generated in the mixture by the irradiation with excitation light L1; thereby, characteristics of the biological sample S3 may be measured. As described above, the excitation light L1 is emitted from the light source 61 of the optical unit 27, and the detection light L2 is detected by the detector 69 of the optical unit 27.

In step S29, the mixture in the first container C1 is continuously irradiated with excitation light L1, and detection light L2 from the first container C1 is continuously detected. The irradiation with excitation light L1 and the detection of detection light L2 can be started before the addition of the second solution S2 in step S28. When the first container C1 in which reaction between the fluorescence indicator and HOCl generated from the biological sample S3 is in progress is irradiated with excitation light L1 having a wavelength of about 480 nm, fluorescence having a wavelength of about 515 nm is generated in the first container C1. In step S29, detection light L2 including fluorescence having a wavelength of about 515 nm is detected. Thereby, myeloperoxidase activity is measured.

After step S29, an evaluation value may be calculated based on the measurement result, and information indicating the measurement result and the evaluation value may be displayed on the display unit 30, or printed by the output unit 50. Additionally, subsequent to step S24 (the sixth step) and step S28 (the fifth step), and prior to step S29 (the seventh step), a step of stirring the mixture in the first container C1 may be performed for example by means of the rotor R1.

As described hereinabove, in the measurement device 1 and the measurement method according to the present embodiment, a first container C1 accommodating therein a first solution S1 that is frozen can be supported by a support unit, and in this state the first solution S1 can be thawed by the heater 25. Thereby, after the first solution S1 is thawed in the vicinity of the subject (in the measurement device 1), the biological sample S3 collected from the subject can be added to the first solution S1. Thus, not only is the quality of the first solution S1 maintained, but also operations such as preparation of the first solution S1 and introduction of the first solution S1 into the first container C1 become unnecessary in the vicinity of the subject.

Thus, by the measurement device 1 and the measurement method according to the present embodiment, the quality of the first solution S1 is maintained, and an operation for measuring characteristics of the biological sample S3 is simplified. Further, in the measurement device 1 and the measurement method according to the present embodiment, the first adjustment unit 21 moves, along the height direction intersecting the liquid surface T of the first solution S1, the first nozzle 13 to which the second container C2 is attached, and thereby adjusts the addition position of the second solution S2 with respect to the liquid surface T of the first solution S1 in the height direction. Thereby, the second solution S2 can be added to the first solution S1 at an appropriate addition position with respect to the liquid surface T of the first solution S1.

Further, in the measurement device 1 according to the present embodiment, when adding the second solution S2 to the first solution S1, the first adjustment unit 21 moves the first nozzle 13 along the height direction such that the distance D2 from the liquid surface T in the height direction of the outlet for the second solution S2 in the second container C2 is 1 mm or less in a range in which the outlet is not in contact with the liquid surface T.

Further, in the measurement method according to the present embodiment, in step S27, when adding the second solution S2 to the first solution S1, the first adjustment unit 21 of the measurement device 1 moves the first nozzle 13 along the height direction such that the distance from the liquid surface T in the height direction of the outlet for the second solution S2 in the second container C2 is 1 mm or less in a range in which the outlet is not in contact with the liquid surface T.

When the second solution S2 is thus discharged from the second container C2 at a distance D2 of 1 mm or less from the liquid surface T of the first solution S1, remaining of the second solution S2 in the second container C2 can be suppressed. Further, an increase in the amount of scattering of the second solution S2, discharged from the second container C2, onto the inner wall surface of the first container C1 can be suppressed. Further, by preventing the outlet for the second solution S2 in the second container C2 from coming into contact with the first solution S1, great disturbance of the liquid surface of the first solution S1 due to addition of the second solution S2 can be suppressed, and disturbance in the detection signal can be suppressed.

In particular, in the case where irradiation of the mixture in the first container C1 with excitation light L1 and detection of detection light L2 from the first container C1 in step S29 are started before the addition of the second solution S2 in step S28, the suppression of scattering of the second solution S2 to the inner wall surface of the first container C1 and the suppression of disturbance of the liquid surface of the first solution S1 become more effective by, in step S27, the first adjustment unit 21 of the measurement device 1 moving the first nozzle 13 along the height direction such that the distance from the liquid surface T in the height direction of the outlet for the second solution S2 in the second container C2 is 1 mm or less in a range in which the outlet is not in contact with the liquid surface T.

Further, the measurement device 1 according to the present embodiment includes a blower 24 for, in the support state, supplying air to the first container C1. The first container C1 includes a light transmission portion Cp that transmits excitation light L1 and detection light L2, and in the support state the blower 24 supplies air to the surface of the light transmission portion Cp.

Further, in the measurement method according to the present embodiment, in step S22, when thawing the first solution S1, the blower 24 of the measurement device 1 is used to supply air to the surface of the light transmission portion Cp that transmits excitation light L1 and detection light L2 in the first container C1.

Thus, by using the blower 24 to supply air to the surface of the transmission portion for excitation light L1 and detection light L2 in the first container C1, the occurrence of dew condensation on the first container C1 during thawing of the first solution S1 can be suppressed, and dew condensation occurring on the first container C1 can be quickly removed. Therefore, the influence of dew condensation on the surface of the light transmission portion Cp in the first container C1 can be quickly reduced, and measurement time can be shortened. The measurement time does not mean only the time during which detection light L2 is monitored in step S29, but means the total time from step S21 to step S29.

Further, in the measurement device 1 according to the present embodiment, the optical unit 27 includes an optical filter 65 for selectively transmitting, out of the detection light L2, detection light L2 incident at angles of incidence equal to or less than a predetermined angle, a lens 68 for condensing detection light L2 transmitted through the optical filter 65, and a detector 69 that detects detection light L2 condensed by the lens 68. Thus, components included in detection light L2 that are influenced by scattering can be removed by the optical filter 65. Further, even when the amount of detection light L2 is reduced due to some components being removed by the optical filter 65, the influence of the reduction in the amount of light on the detection result can be suppressed by condensing the detection light L2 with the lens 68 in a stage subsequent to the optical filter 65.

The optical filter 65 is preferably provided in a stage prior to optical filters 66 and 67 that transmit some wavelength components of the detection light L2. In this case, only straight light transmitted through the optical filter 65 is incident on the optical filters 66 and 67, and scattered light can be reliably reduced.

Further, the measurement device 1 according to the present embodiment includes a second adjustment unit 22 for, in the support state, adjusting the addition position of the biological sample S3 (In the first modified example, the biological sample S3 and the fluorescence indicator (hereinafter, the same)) with respect to the first solution S1 in the first container C1. A third container C3 accommodating the biological sample S3 is attached to the second addition unit 42, and the second addition unit 42 includes a second nozzle 15 for discharging the biological sample S3 from the third container C3 to the first container C1. The second adjustment unit 22 moves the second nozzle 15 along the height direction, and thereby adjusts the addition position of the biological sample S3 in the height direction.

Further, the measurement method according to the present embodiment includes step S21 of, before the sixth step, attaching a third container accommodating therein the biological sample to a second nozzle of the measurement device, and step S23 of, after step S21 and before step S24, adjusting the addition position of the biological sample S3 with respect to the first solution S1 in the first container C1. In step S23, the second adjustment unit 22 of the measurement device 1 moves the second nozzle 15 along the height direction, and thereby adjusts the addition position of the biological sample S3 with respect to the first solution S1 in the height direction.

Thus, the biological sample S3 can be added to the first solution S1 at an appropriate addition position with respect to the first solution S1.

Further, in the measurement device 1 according to the present embodiment, the second adjustment unit 22 moves the second nozzle 15 along the height direction such that the outlet for the biological sample S3 in the third container C3 is located in the first solution S1.

Further, in the measurement method according to the present embodiment, in step S23, the second adjustment unit 22 of the measurement device 1 moves the second nozzle 15 along the height direction such that the outlet for the biological sample S3 in the third container C3 is located in the first solution S1.

In this case, the biological sample S3 can be discharged from the third container C3 in the interior of the first solution S1.

Further, in the measurement device 1 according to the present embodiment, when adding the biological sample S3 to the first solution S1, the second addition unit 42 repeatedly performs the discharge of the biological sample S3 from the third container C3 by means of the second nozzle 15 and the introduction of the first solution S1 into the third container C3 by means of the second nozzle 15.

Further, in the measurement method according to the present embodiment, in step S24, when adding the biological sample S3 to the first solution S1, the discharge of the biological sample S3 from the third container C3 by means of the second nozzle 15 and the introduction of the first solution S1 into the third container C3 by means of the second nozzle 15 are repeatedly performed.

Thus, by performing pipetting at the third container C3 by means of the second nozzle 15, the biological sample S3 in the third container C3 can be reliably added to the first solution S1. In particular, by increasing the amount of ejection from the third container C3 in the last time of discharge in pipetting, remaining of the biological sample S3 in the third container C3 can be reliably suppressed.

Further, in the present embodiment, the second solution S2 does not contain a buffer solution. Thus, the total amount of the second solution S2 in the second container C2 is reduced, and therefore the influence of remaining of liquid in the second container C2 on measurement accuracy is increased. Therefore, the effect of suppressing remaining of liquid by discharging the second solution S2 from the second container C2 at a distance D2 of 1 mm or less from the liquid surface T of the first solution S1 as described above is larger.

The above embodiment is what is described for one aspect of the present disclosure. Therefore, the present disclosure is not limited to the above embodiment, and can be arbitrarily modified.

For example, the subject is not limited to a human body, and may be, for example, an animal or the like. Further, although in the above embodiment an example in which the biological sample S3 is blood of the subject is described, the biological sample S3 may be, for example, another body fluid or the like of the subject. The biological sample S3 may be, for example, saliva, perfusate, lacrimal fluid, sweat, urine, or the like of the subject. Although an example of the activity of a neutrophil is described as characteristics of the biological sample S3, the characteristics of the biological sample S3 may be, for example, the activity of a tissue cell such as a monocyte, an eosinophil, a basophil, a B cell, a T cell, an NK cell, or a vascular endothelial cell.

Further, although in the above embodiment an example in which the first solution S1 contains a fluorescence indicator is described, the first solution S1 may contain a chemiluminescence indicator. The chemiluminescence indicator reacts with a component (for example, superoxide or the like) generated from the biological sample. The chemiluminescence indicator is, for example, MCLA (2-Methyl-6-(4-methoxyphenyl)-3,7-dihydroimidazo[1,2-a]pyrazin-3-one) or the like. A commercially available product may be used as the chemiluminescence indicator. In the first container C1 in which reaction between superoxide generated from the biological sample S3 and a chemiluminescence indicator is in progress, chemiluminescence having a maximum emission wavelength of about 465 nm is generated. Therefore, in step S29, chemiluminescence having a maximum emission wavelength of about 465 nm may be detected. Thereby, superoxide production activity is measured. In the first modified example, the first solution S1 may not contain the chemiluminescence indicator, and the chemiluminescence indicator may be accommodated in the third container C3.

Further, although in the above embodiment an example in which each of the second container C2 and the third container C3 is a pipette tip is described, at least one of the second container C2 and the third container C3 may be, for example, a capillary nozzle, a dropper, a whole pipette, a Komagome pipette, a measuring pipette, a hematocrit capillary, a syringe, or the like.

Further, the first solution S1 may contain, as an indicator that reacts with hypochlorous acid (or a halogen equivalent thereof) produced by myeloperoxidase, taurine/TNB (see J. Clin. Invest., Vol. 70, pp. 598-607, 1982), 8-amino-5-chloro-7-phenylpyrido[3,4-d]pyridazine-1,4-(2H,3H)dione (L-012, see Anal. Biochem., Vol. 271(1), pp. 53-58, 1999), or the like, for example. The above indicators may also, like the above-described fluorescence indicator and chemiluminescence indicator, be accommodated in the third container C3 without being included in the first solution S1.

Further, the first solution S1 may contain, as an indicator that reacts with superoxide, 2-methyl-6-phenyl-3,7-dihydroimidazo[1,2-a]pyrazin-3-one (CLA), 2-methyl-6-p-methoxyphenylethynylimidazopyrazinone (MPEC), an indocyanine-type imidazopyrazinone compound (NIR-CLA), 2-[2,4,5,7-tetrafluoro-6-(2-nitro-4,5-dimethoxyphenylsulfonyloxy)-3-oxo-3H-xanthene-9-yl]benzoic acid (BES-So), or the like, for example. The above indicators may also, like the above-described fluorescence indicator and chemiluminescence indicator, be accommodated in the third container C3 without being included in the first solution S1.

Further, the second solution S2 may contain, as a stimulant, opsonized zymosan (OZ), for example.

Further, although in the above embodiment an example in which a first solution S1 prepared in advance outside the first container C1 is accommodated in the first container C1 is described, the first solution S1 may be prepared in the interior of the first container C1. For example, the plurality of reagents may be introduced into the first container C1 in a state of being separated from each other. Further, the plurality of reagents may be sequentially or simultaneously introduced into the first container C1.

Here, when the total of the amount of the buffer solution necessary to prepare the first solution S1 and the amount of the buffer solution necessary to prepare the second solution S2 is set as a specific prescribed amount, the prescribed amount of the buffer solution may be readied in step S12 of preparing the first solution S1. In this case, in step S12, a first solution S1 containing the buffer solution necessary to prepare the first solution S1 and the buffer solution necessary to prepare the second solution S2 (that is, containing the prescribed amount of the buffer solution) may be prepared. Then, in step S13, the first solution S1 containing the prescribed amount of the buffer solution may be introduced into the first container C1. In other words, also the necessary amount of the buffer solution to prepare the second solution S2 may be introduced into the first container C1 in advance in step S13. Further, the necessary amount of the buffer solution to prepare the second solution S2 may be added to the first container C1 before the addition of the second solution S2 to the first container C1 in step S28. In these cases, the second solution S2 in the second container C2 does not contain the buffer solution. In step S12, only the necessary amount of the buffer solution to prepare the first solution S1 may be readied, and a first solution S1 containing this exclusive amount of the buffer solution may be prepared. Further, the amount of the buffer solution contained in the first solution S1 can be adjusted depending on whether the second solution S2 contains the buffer solution or not, as appropriate.

Further, it is not essential to, when adding the second solution S2 to the first solution S1, move the first nozzle 13 along the height direction such that the distance D2 from the liquid surface T in the height direction of the outlet for the second solution S2 in the second container C2 is 1 mm or less in a range in which the outlet is not in contact with the liquid surface T. That is, the first adjustment unit 21 may adjust the distance D2 in a range other than the above range.

Further, the measurement device 1 may not include the blower 24, and thus may not supply air to the light transmission portion Cp of the first container C1 by means of the blower 24. Further, the optical unit 27 may not include an optical filter 65 for selectively transmitting, out of the detection light L2, detection light incident at angles of incidence equal to or less than a predetermined angle.

Further, the measurement device 1 may not include the second adjustment unit 22, and even in the case where the measurement device 1 includes the second adjustment unit 22, it is not essential to use the second adjustment unit 22 to move the second nozzle 15 along the height direction such that the outlet for the biological sample S3 in the third container C3 is located in the first solution S1. Further, the second adjustment unit 22 may not perform pipetting at the third container C3.

What is claimed is:

1. A measurement device used for measurement of a characteristic of a biological sample,
the measurement device comprising:
a support unit for supporting a first container configured to accommodate therein a first solution containing an indicator and frozen, the indicator being to react with a component generated from a biological sample;
a heater configured to heat the first solution in the first container in a support state where the support unit supports the first container;
a first addition unit configured to, in the support state, add a second solution containing a stimulant to the first solution in the first container, the stimulant being to activate a function of the biological sample;
a second addition unit configured to, in the support state, add the biological sample to the first solution in the first container;
a first adjustment unit configured to, in the support state, adjust an addition position of the second solution with respect to a liquid surface of the first solution in the first container; and
an optical unit configured to, in the support state, perform irradiation of a mixture of the first solution thawed, and the second solution and the biological sample added to the first solution with excitation light and detection of detection light including fluorescence generated in the mixture by irradiation with the excitation light,
wherein the first addition unit includes a first nozzle to which a second container accommodating the second solution is attached, and the first nozzle is configured to discharge the second solution from the second container to the first solution, and
the first adjustment unit moves the first nozzle along a height direction intersecting the liquid surface and thereby adjusts an addition position of the second solution with respect to the liquid surface in the height direction.

2. A measurement device used for measurement of a characteristic of a biological sample,
the measurement device comprising:
a support unit for supporting a first container configured to accommodate therein a frozen first solution for diluting the biological sample;
a heater configured to heat the first solution in the first container in a support state where the support unit supports the first container;
a first addition unit configured to, in the support state, add a second solution containing a stimulant to the first solution in the first container, the stimulant being to activate a function of the biological sample;
a second addition unit configured to, in the support state, add the biological sample and an indicator being to react with a component generated from the biological sample to the first solution in the first container;
a first adjustment unit configured to, in the support state, adjust an addition position of the second solution with respect to a liquid surface of the first solution in the first container; and
an optical unit configured to, in the support state, perform irradiation of a mixture of the first solution thawed, and the second solution, the biological sample, and the indicator added to the first solution with excitation light and detection of detection light including fluorescence generated in the mixture by irradiation with the excitation light,
wherein the first addition unit includes a first nozzle to which a second container accommodating the second solution is attached, and the first nozzle is configured to discharge the second solution from the second container to the first solution, and
the first adjustment unit moves the first nozzle along a height direction intersecting the liquid surface and thereby adjusts an addition position of the second solution with respect to the liquid surface in the height direction.

3. The measurement device according to claim 2, wherein,
when adding the second solution to the first solution, the first adjustment unit moves the first nozzle along the height direction such that a distance from the liquid surface in the height direction of an outlet for the second solution in the second container is 1 mm or less in a range in which the outlet is not in contact with the liquid surface.

4. The measurement device according to claim 2,
including a blower configured to, in the support state, supply air to the first container,
wherein the first container includes a light transmission portion configured to transmit the excitation light and the detection light, and
the blower supplies the air to a surface of the light transmission portion in the support state.

5. The measurement device according to claim 2, wherein
the optical unit includes:
an optical filter configured to selectively transmit, out of the detection light from the mixture, the detection light incident at angles of incidence equal to or less than a predetermined angle;
a lens configured to condense the detection light transmitted through the optical filter; and
a detector configured to detect the detection light condensed by the lens.

6. The measurement device according to claim 2,
including a second adjustment unit configured to, in the support state, adjust an addition position of the biological sample and the indicator with respect to the first solution in the first container,
wherein the second addition unit includes a second nozzle to which a third container accommodating the biological sample and the indicator is attached, and the second nozzle configured to discharge the biological sample from the third container to the first container, and
the second adjustment unit moves the second nozzle along the height direction and thereby adjusts the addition position of the biological sample and the indicator in the height direction.

7. The measurement device according to claim 6, wherein
the second adjustment unit moves the second nozzle along the height direction such that an outlet for the biological sample and the indicator in the third container is located in the first solution.

8. The measurement device according to claim 7, wherein,
when adding the biological sample and the indicator to the first solution, the second addition unit repeatedly performs discharge of the biological sample and the indicator from the third container by means of the second nozzle and introduction of the first solution into the third container by means of the second nozzle.

9. A measurement method for measuring a characteristic of a biological sample by using a measurement device,
the measurement method comprising:
a first step of causing a support unit of the measurement device to support a first container accommodating therein a first solution containing an indicator and frozen, the indicator being to react with a component generated from a biological sample;
a second step of, after the first step, using a heater of the measurement device to thaw the first solution in the first container;
a third step of attaching, to a first nozzle of the measurement device, a second container accommodating therein a second solution containing a stimulant to activate a function of the biological sample;
a fourth step of, after the second step and the third step, adjusting an addition position of the second solution with respect to a liquid surface of the first solution in the first container;
a fifth step of, after the fourth step, adding the second solution to the first solution in the first container;
a sixth step of, after the second step, adding the biological sample to the first solution in the first container; and
a seventh step of, after the fifth step and the sixth step, using an optical unit of the measurement device to perform irradiation of a mixture of the first solution, and the biological sample and the second solution added to the first solution with excitation light and detection of detection light including fluorescence generated in the mixture by irradiation with the excitation light,
wherein, in the fourth step, a first adjustment unit of the measurement device moves the first nozzle along a height direction intersecting a liquid surface of the first solution, and thereby adjusts an addition position of the second solution with respect to the liquid surface in the height direction.

10. A measurement method for measuring a characteristic of a biological sample by using a measurement device,
the measurement method comprising:
a first step of causing a support unit of the measurement device to support a first container accommodating therein a frozen first solution for diluting the biological sample;
a second step of, after the first step, using a heater of the measurement device to thaw the first solution in the first container;
a third step of attaching, to a first nozzle of the measurement device, a second container accommodating therein a second solution containing a stimulant to activate a function of the biological sample;
a fourth step of, after the second step and the third step, adjusting an addition position of the second solution with respect to a liquid surface of the first solution in the first container;
a fifth step of, after the fourth step, adding the second solution to the first solution in the first container;
a sixth step of, after the second step, adding the biological sample and an indicator being to react with a component generated from the biological sample to the first solution in the first container; and
a seventh step of, after the fifth step and the sixth step, using an optical unit of the measurement device to perform irradiation of a mixture of the first solution, and the biological sample, the indicator, and the second solution added to the first solution with excitation light and detection of detection light including fluorescence generated in the mixture by irradiation with the excitation light,
wherein, in the fourth step, a first adjustment unit of the measurement device moves the first nozzle along a height direction intersecting a liquid surface of the first solution, and thereby adjusts an addition position of the second solution with respect to the liquid surface in the height direction.

11. The measurement method according to claim 10, wherein,
in the fourth step, when adding the second solution to the first solution, the first adjustment unit of the measurement device moves the first nozzle along the height direction such that a distance from the liquid surface in the height direction of an outlet for the second solution in the second container is 1 mm or less in a range in which the outlet is not in contact with the liquid surface.

12. The measurement method according to claim 10, wherein,
in the second step, when thawing the first solution, a blower of the measurement device is used to supply air to a surface of a light transmission portion configured to transmit the excitation light and the detection light in the first container.

13. The measurement method according to claim 10,
including:
an eighth step of, before the sixth step, attaching a third container accommodating therein the biological sample and the indicator to a second nozzle of the measurement device; and
a ninth step of, after the eighth step and before the sixth step, adjusting an addition position of the biological sample and the indicator with respect to the first solution in the first container,
wherein, in the ninth step, a second adjustment unit of the measurement device moves the second nozzle along the height direction, and thereby adjusts an addition position of the biological sample and the indicator with respect to the first solution in the height direction.

14. The measurement method according to claim 13, wherein,
in the ninth step, the second adjustment unit of the measurement device moves the second nozzle along the height direction such that an outlet for the biological sample and the indicator in the third container is located in the first solution.

15. The measurement method according to claim 14, wherein,
in the sixth step, when adding the biological sample and the indicator to the first solution, discharge of the biological sample and the indicator from the third container by means of the second nozzle and introduction of the first solution into the third container by means of the second nozzle are repeatedly performed.
